(19) United States
(12) Patent Application Publication
(10) Pub. No.: US 2022/0391387 A1
(43) Pub. Date: Dec. 8, 2022

(54) Integrated Data Compliance Monitoring Platform

(71) Applicant: SleepSafe Drivers, Inc., Lenexa, KS (US)

(72) Inventors: Adrian Knight, Lenexa, KS (US); Mary Convey, Tierra Verde, FL (US)

(21) Appl. No.: 17/342,432

(22) Filed: Jun. 8, 2021

(51) Int. Cl. G06F 16/2455; G16H 10/60; G06F 21/60

(52) U.S. Cl. CPC G06F 16/2455; G16H 10/60; G06F 21/602

(57) ABSTRACT

Disclosed are systems and methods for integrating data and compliance monitoring. Certain embodiments provide a method includes loading an application programming interface preselected to generate a query. The query is sent via a network to a database requesting data for a patient. The data for the patient is received from the database via the network. The data associated is stored with the patient in a compliance database. The data for the patient is compared to a rule set to determine a compliance measurement. An output that includes the compliance measurement is generated.

### TECHNICAL FIELD

The subject matter described herein relates to systems and methods for integrating data compliance and monitoring to help manage patient data, compliance, and safety.

### BACKGROUND

Sleep disorders are conditions that affect sleep quality, timing, or duration of a person's sleep and thus impact the ability to properly function when awake. For some occupations that require attentiveness for prolonged periods of time, sleep disorders can increase the risk of accidents to the person, surrounding people, and property. Job activities, such as operating dangerous machinery, driving vehicles, flying airplanes, operating trains, security, military and medical jobs, and mining and oil work, each require focus that can be impaired by a lack of sleep.

Fortunately, many sleep disorders are treatable. Continuous Positive Airway Pressure (CPAP) systems provide a supply of air or breathable gas from a blower to a patient wearing a full-face or nasal mask, or nasal prongs, while they sleep. However, wearing a mask while sleeping can be cumbersome, and along with other drawbacks, can lead to patients not using their CPAP system resulting in low compliance rates. By not using their CPAP systems, patients are at greater risk of workplace performance issues created by a lack of sleep. Minimizing workplace performance issues is important to employers who want to keep all employees safe, as well as the people who interact with the employees.

### SUMMARY

Aspects of the current subject matter relate inter alia to systems and methods for integrating data and compliance monitoring.

Consistent with some aspects of the current subject matter, a method for integrating data and compliance monitoring is disclosed. The method includes loading an application programming interface (API) preselected to generate a query. The query is sent via a network to a database requesting data for a patient. The data for the patient is received from the database via the network. The data associated is stored with the patient in a compliance database. The data for the patient is compared to a rule set to determine a compliance measurement. An output that includes the compliance measurement is generated.

Aspects of the current subject matter also include a method for integrating data and compliance monitoring that includes loading a first API preselected to generate a first query. A second API preselected to generate a second query is also loaded. The first query is sent to a first database for data associated with a first patient and the second query is sent to the second database for data associated with a second patient. The data associated with the first patient and the data associated with the second patient are received. The data associated with the first patient and the data associated with the second patient are stored in a compliance database. The data associated with the first patient and/or the data associated with the second patient are compared to a rule set to determine a compliance measurement. An output that includes the compliance measurement is generated.

Aspects of the current subject matter also include a system for implementing the aforementioned methods. The system for integrating data and compliance monitoring includes a server configured for loading a first application programming interface (API) preselected to generate a first query and for loading a second API preselected to generate a second query. The first query is sent to a first database for data associated with a first patient and the second query is sent to the second database for data associated with a second patient. The data associated with the first patient and the data associated with the second patient are received. The data associated with the first patient and the data associated with the second patient are stored in a compliance database. The data associated with the first patient and/or the data associated with the second patient are compared to a rule set to determine a compliance measurement. An output device generates an output that includes the compliance measurement is generated.

Aspects of the current subject matter also include a non-transitory processor-readable medium containing processor executable instructions for implementing the aforementioned methods. The processor executable instructions include loading a first application programming interface (API) preselected to generate a first query and loading a second API preselected to generate a second query. The processor executable instructions include sending the first query to a first database for data associated with a first patient and sending the second query to the second database for data associated with a second patient. The processor executable instructions also include receiving the data associated with the first patient and receiving the data associated with the second patient. The processor executable instructions further include storing the data associated with the first patient and storing the data associated with the second patient in a compliance database, and comparing the data associated with the first patient and/or the data associated with the second patient to a rule set to determine a compliance measurement. The processor executable instructions include generating an output that includes the compliance measurement.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter of this disclosure are contemplated as being part of the inventive subject matter disclosed herein. It should also be appreciated that terminology explicitly employed herein should be accorded a meaning most consistent with the particular concepts disclosed herein.

### BRIEF DESCRIPTION OF THE DRAWINGS

The skilled artisan will understand that the drawings primarily are for illustrative purposes and are not intended to limit the scope of the inventive subject matter described herein. The drawings are not necessarily to scale, and in some instances, various aspects of the subject matter disclosed herein may be shown exaggerated or enlarged in the drawings to facilitate an understanding of different features. In the drawings:

FIG. 1 illustrates a diagram of a system for integrating data and compliance monitoring consistent with implementations of the current subject matter;

FIG. 2 illustrates a block diagram of a method for integrating data and compliance monitoring consistent with implementations of the current subject matter;

FIG. 3 illustrates a block diagram of a method for integrating data and compliance monitoring consistent with implementations of the current subject matter;

FIG. 4 illustrates a compliance dashboard (e.g., Client Dashboard) consistent with implementations of the current subject matter;

FIG. 5 illustrates a compliance dashboard (e.g., All Dashboard) consistent with implementations of the current subject matter;

FIG. 6 illustrates a compliance dashboard consistent with implementations of the current subject matter;

FIG. 7 illustrates a form for entering text associated with a patient consistent with implementations of the current subject matter;

FIG. 8 illustrates an email address output form consistent with implementations of the current subject matter; and

FIG. 9 illustrates a compliance report output form consistent with implementations of the current subject matter.

### DETAILED DESCRIPTION

In the following description, certain specific details are set forth in order to provide a thorough understanding of various embodiments. However, one skilled in the art will understand that the disclosure may be practiced without these details. In other instances, well-known structures have not been shown or described in detail to avoid unnecessarily obscuring descriptions of the embodiments. Unless the context requires otherwise, throughout the specification and claims which follow, the word “comprise” and variations thereof, such as, “comprises” and “comprising” are to be construed in an open, inclusive sense, that is, as “including, but not limited to.” Further, headings provided herein are for convenience only and do not interpret the scope or meaning of the claimed disclosure.

Reference throughout this specification to “one embodiment” or “an embodiment” means a particular feature, structure or characteristic described in connection with the embodiment is included in at least one embodiment. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments. Also, as used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise.

Modern CPAP systems can have sensors and software that monitors the patient's sleep not only while using the system, but also can tell when the CPAP system is not being used. This information (e.g., compliance or usage data), for example, can be used by insurance companies to determine whether to cover the cost of CPAP systems. This information is also of interest to employers that monitor employees that are known to suffer from sleep related disorders, among other entities.

There are many manufacturers of CPAP systems. It can be cumbersome and burdensome individually log into each CPAP manufacturer's database to retrieve a patient's compliance data. There is a need for systems and methods of integrating the data from the many manufacturers of CPAP systems and monitoring the data for compliance that is measured against one or more rule sets.

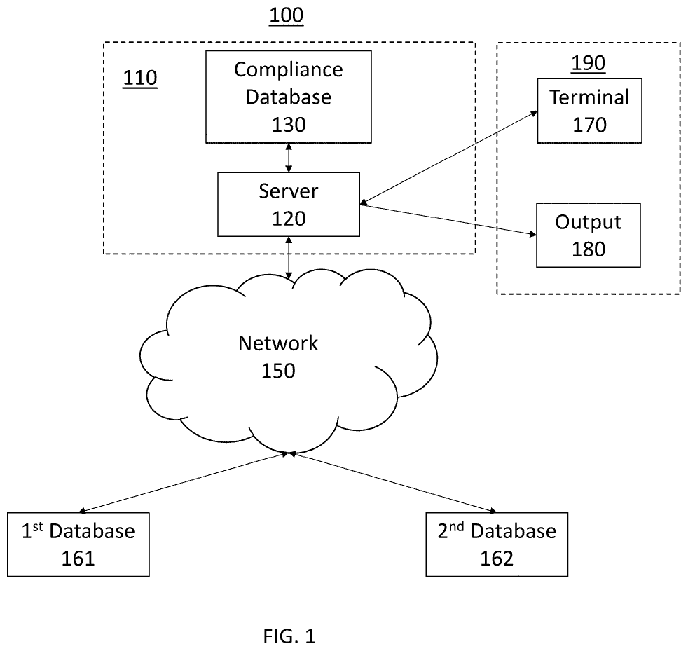

Aspects of the current subject matter provide an integrated data compliance monitoring platform that is able to compile and analyze data from a one or more source databases. In this manner, the platform allows an interested party to quickly determine individual and group compliance as measured against one or more rule sets. FIG. 1 illustrates a diagram of an exemplary system 100 including an integrated data compliance monitoring platform consistent with implementations of the current subject matter. A computer system 110 includes a server 120 and a compliance database 130. In alternative embodiments, the server and compliance database are components of two separate computer systems. In these embodiments, the server and compliance database are communicatively coupled over a network or a direct connection. The server 120 is communicatively coupled to a first database 161, a second database 162, and a third database 163 over a network 150. While exemplary system 100 is illustrated as having three databases, it should be recognized that the exemplary system 100 can have any natural number of databases. The server 120 is also communicatively coupled to a client system 190 that includes a terminal 170 and an output device 180.

The server 120 is configured for loading a first application programming interface (API) preselected to generate a first query. The server 120 is also configured for loading a second API preselected to generate a second query. The server 120 is also capable of loading any number of additional APIs that are preselected to generate additional queries. The server 120 sends the first query to a first database 161 for data associated with a first patient and sends the second query to the second database 163 for data associated with a second patient via network 150. Each API was developed based on the respective requirements of the first database 161 and second database 162. In this manner, the API generates a query that is compatible with the database. Preselection of the API ensures that the given query that is compatible with the given database.

The server 120 receives the data associated with the first patient from the first database 161 and receives the data associated with the second patient from the second database 162 via network 150. The data associated with the first patient and the data associated with the second patient is stored by server 120 in a compliance database 130. The data associated with the first patient and/or the data associated with the second patient is compared by the server 120 to a rule set to determine a compliance measurement. The client system 190 includes an output device 180. An output device 180 is configured for receiving data from the server 120 and generating an output that includes the compliance measurement.

The first query and/or the second query can contain a patient identification (ID) number, a patient name, a date, a date range, a password, a device ID, an authentication token, and/or the like. The first query and/or the second query can also contain a request for device usage, activation date, activation status, employer name, and/or the like. The first query and/or the second query can occur at a specified interval, upon demand, or at a random time. The specified interval can be hourly, daily, weekly, monthly, and/or at any other given period of time. The data associated with the first patient and/or the data associated with the second patient can contain a patient ID number, a patient name, a date, a date range, a password, a device ID, an authentication token, device usage, activation date, activation status, employer name, and/or the like. The data associated with the first patient and/or the data associated with the second patient can be encrypted prior to receiving the data via the network 150. In some embodiments, the first database 161 and/or the second database 162 are remote databases accessible via the network 150. The data can also be encrypted when the data associated with the first patient and/or the data associated with the second patient is stored in the compliance database 130. The compliance database can be a persistent data store. The compliance database can be compliant with Health Insurance Portability and Accountability Act (HIPAA) of 1996. The compliance measurement can be pass or fail value based on a minimum threshold.

The output device 180 can be a display, printer, the first server, and/or a second server. The output device 180 generates an output, which can be a text message. The output can also be an email message. For example, if a patient falls below a 70% compliance rate, an email message, a text message, phone call, and/or other communication can be generated and sent to the patient and/or the employer. The output can be rendered on a graphical user interface, for example, an electronic display. The output can be a dashboard rendered on a graphical user interface. The output can be an automated resupply call. The output can also be a report. The report can include data associated with a plurality of patients. The compliance measurement can be date and/or time stamped. The data can include a null value indicating that data is missing. For example, if data is missing for a pre-determined period of time (such as a period of 7 days), an email message, phone message, and/or a text message can be generated and sent to the patient, monitoring company, and/or the employer to make them aware of the situation.

The exemplary system 100 can further include a terminal 170 for entering text associated with the first patient and/or the second patient. The text can be stored in the compliance database. The exemplary system 100 can further include receiving data from a piece of machinery by the server 120 via the network 150. The piece of machinery is associated with the first patient and/or the second patient. The data from the piece of machinery can be stored in the compliance database 130. The piece of machinery can be, for example, a vehicle. The vehicle can have a gross vehicle weight rating of 26,001 or more pounds, such as a commercial truck.

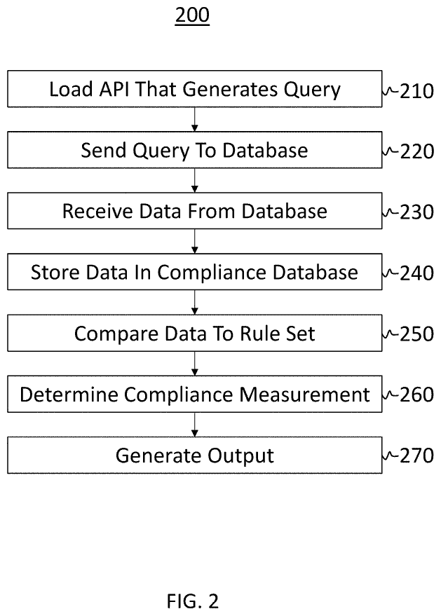

FIG. 2 illustrates a block diagram of a method 200 for integrating data and compliance monitoring consistent with implementations of the current subject matter. The method 200 includes loading an application programming interface (API) that has been preselected to generate a query in step 210. In step 220, the query is sent via a network to a database requesting data for a patient. The data for the patient from the database is received via the network in step 230. The data associated with the patient is stored in a compliance database in step 240. In step 250, the data for the patient is compared to a rule set, and in step 260 a compliance measurement is determined. An output including the compliance measurement is generated in step 270.

The query of method 200 can contain a patient ID number, a patient name, a date, a date range, a password, a device ID, an authentication token, and/or the like. The query can contain a request for device usage, activation date, activation status, employer name, and/or the like. The query can occur at a specified interval, upon demand, or at a random time. The specified interval can be hourly, daily, weekly, monthly, and/or at any other given period of time. The data can contain a patient ID number, a patient name, a date, a date range, a password, a device ID, an authentication token, device usage, activation date, activation status, employer name, and/or the like. The data can be encrypted prior to receiving the data for the patient from the database via the network. The database is a remote database accessible via the network. The data can be encrypted when the data associated with the patient is stored in the compliance database. The compliance database can be a persistent data store. The compliance database can be HIPAA compliant. The compliance measurement can be a pass or fail value based on a minimum threshold. The output can be a text message. The output can be an email message or phone call. For example, if a patient falls below a 70% compliance rate, an email message, phone message, and/or a text message can be generated and sent to the patient and/or the employer. The output can be rendered on a graphical user interface. The output can be a dashboard rendered on a graphical user interface. The output can be an automated resupply call. The output can be a report. The report can include data associated with a plurality of patients. The compliance measurement can be date and/or time stamped. The data can include a null value indicating that data is missing. For example, if data is missing for a pre-determined period of time (such as a period of 7 days), an email message, phone message, and/or a text message can be generated and sent to the patient, monitoring company and/or the employer to make them aware of the situation.

The method 200 can further include entering text associated with the patient and storing the text in the compliance database. The method 200 can also further include receiving data from a piece of machinery. The piece of machinery is associated with the patient. The data from the piece of machinery can be stored in the compliance database. The piece of machinery can be, for example, a vehicle. The vehicle can have a gross vehicle weight rating of 26,001 or more pounds, such as a commercial truck.

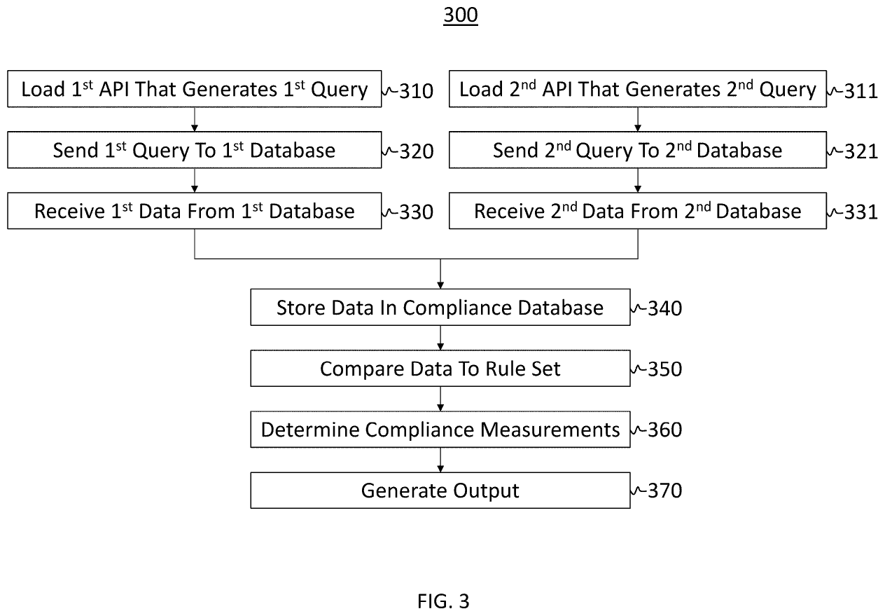

FIG. 3 illustrates a block diagram of a method 300 for integrating data and compliance monitoring consistent with implementations of the current subject matter. The method 300 includes loading a first application programming interface (API) that has been preselected to generate a first query in step 310, and also includes loading a second API that has been preselected to generate a second query in step 311. In step 320, the first query is sent via a network to a first database requesting data for a first patient, and in step 321, the second query is sent via a network to a second database requesting data for a second patient. The data for the first patient from the first database is received via the network in step 330, and the data for the second patient from the second database is received via the network in step 331. The first data associated with the first patient and the second data associated with the second patient is stored in a compliance database in step 340. In step 350, the data for the first patient and the data for the second patient are compared to a rule set, and in step 360 compliance measurements are determined. An output including the compliance measurements is generated in step 370.

The first and/or the second query of method 300 can contain a patient ID number, a patient name, a date, a date range, a password, a device ID, an authentication token, and/or the like. The first and/or the second query can contain a request for device usage, activation date, activation status, employer name, and/or the like. The first and/or the second query can occur at a specified interval, upon demand, or at a random time. The specified interval can be hourly, daily, weekly, monthly, and/or at any other given period of time. The first and/or the second data can contain a patient ID number, a patient name, a date, a date range, a password, a device ID, an authentication token, device usage, activation date, activation status, employer name, and/or the like. The first and/or the second data can be encrypted prior to receiving the first and/or the second data for the patient from the first and/or the second database via the network. The first and/or the second database is a remote database accessible via the network. The first and/or the second data can be encrypted when the first and/or the second data associated with the first and/or the second patient is stored in the compliance database. The compliance database can be a persistent data store. The compliance database can be HIPAA compliant. The compliance measurements can be pass or fail values based on a minimum threshold. The output can be a text message. The output can be an email message or a phone message. For example, if the first and/or the second patient falls below a 70% compliance rate, an email message, phone message, and/or a text message can be generated and sent to the first and/or the second patient and/or an employer. The output can be rendered on a graphical user interface. The output can be a dashboard rendered on a graphical user interface. The output can be an automated resupply call. The output can be a report. The report can include data associated with a plurality of patients. The compliance measurement can be date and/or time stamped. The data can include a null value indicating that data is missing. For example, if data is missing for a pre-determined period of time (such as a period of 7 days), an email message, phone message, and/or a text message can be generated and sent to the first and/or the second patient and/or the employer to make them aware of the situation.

The method 300 can further include entering text associated with the first and/or the second patient, and storing the text in the compliance database. The method 300 can also further include receiving data from a piece of machinery. The piece of machinery is associated with the first and/or the second patient. The data from the piece of machinery can be stored in the compliance database. The piece of machinery can be, for example, a vehicle. The vehicle can have a gross vehicle weight rating of 26,001 or more pounds, such as a commercial truck.

In some embodiments, a non-transitory processor-readable medium can contain processor executable instructions for integrating data and compliance monitoring according to method 200. In other embodiments, a non-transitory processor-readable medium can contain processor executable instructions for integrating data and compliance monitoring according to method 300.

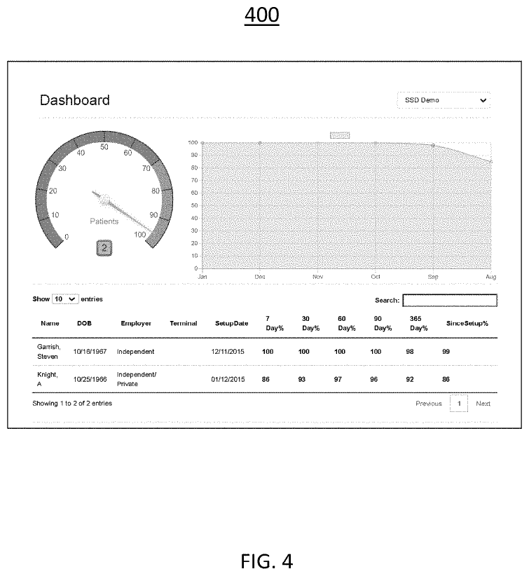

FIG. 4 illustrates a compliance dashboard 400 (e.g., Client Dashboard) consistent with implementations of the current subject matter. The compliance dashboard 400 shows the integrated data for patients of a particular employer or fleet. The comparison data presented can be sorted by name, date of birth (DOB), setup date, specific interval compliance, and elapse time since setup. A graph shown in the upper right portion of the compliance dashboard 400 shows a trending compliance percentage on a month-to-month interval. A drop-down menu allows a user to select the maximum number of entries presented and a search box allows the user to retrieve specific comparison data. An analogue speedometer representing the average patient compliance is shown in the upper left portion of the compliance dashboard 400 with a value of 70% to 100% displayed in green (e.g., compliant) and all other values in red (e.g., noncompliant). The speedometer displays the number of patients used for the measurement (e.g., 2) and the average patient compliance can be determined over a rolling 30-day interval. The compliance dashboard 400 can be, for example, outputted to a display such as terminal 170, printed in a report, or embedded in an email message.

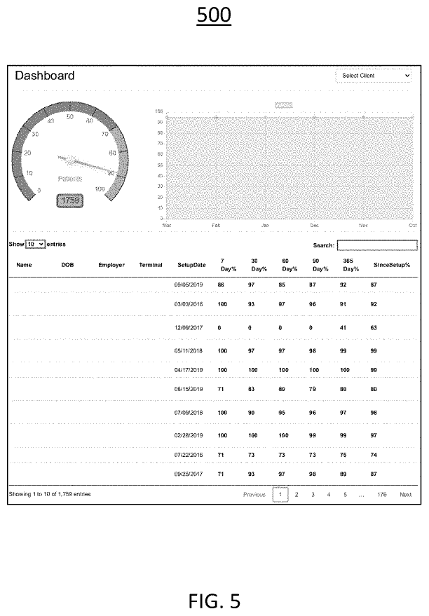

FIG. 5 illustrates a compliance dashboard 500 (e.g., All Dashboard) consistent with implementations of the current subject matter. The compliance dashboard 500 shows the integrated data for multiple patients of multiple employers or multiple fleets. The comparison data presented can be sorted by name, DOB, employer, terminal, setup date, specific interval compliance, and elapse time since setup. A graph shown in the upper right portion of the compliance dashboard 500 shows a trending compliance percentage on a month-to-month interval. A drop-down menu allows a user to select the maximum number of entries presented and a search box allows the user to retrieve specific comparison data. An analogue speedometer representing the average patient compliance is shown in the upper left portion of the compliance dashboard 700 with a value of 70% to 100% displayed in green (e.g., compliant) and all other values in red (e.g., noncompliant). The speedometer displays the number of patients used for the measurement (e.g., 1759) and the average patient compliance can be determined over a rolling 30-day interval. The compliance dashboard 500 can be, for example, outputted to a display such as terminal 170, printed in a report, or embedded in an email message.

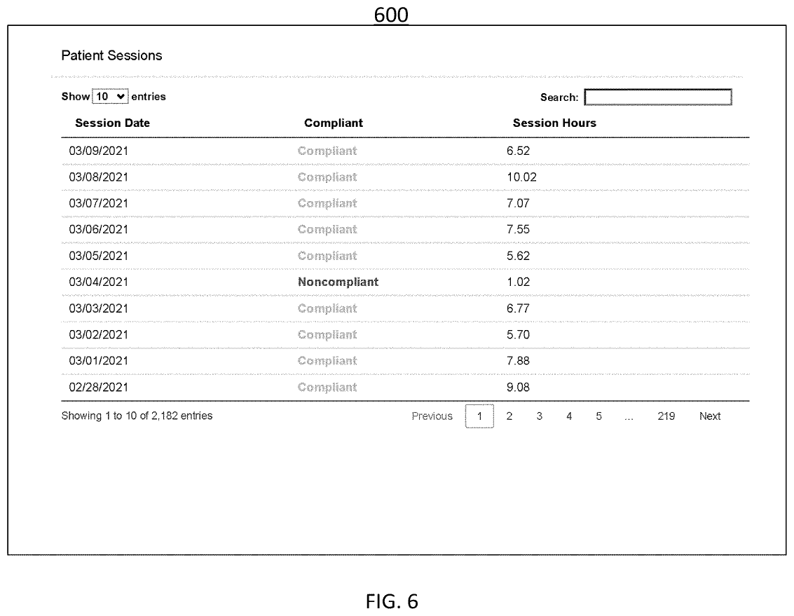

FIG. 6 illustrates a compliance dashboard 600 consistent with implementations of the current subject matter. Compliance dashboard 600 is another view of compliance dashboard 500. In this compliance dashboard 600, the comparison data presented can be sorted by session date, compliant/noncompliant, and session hours. The “compliant/noncompliant” text can be color coded for quick identification with “compliant” being in green, and “noncompliant” being in red. A drop-down menu allows a user to select the maximum number of entries presented and a search box allows the user to retrieve specific comparison data.

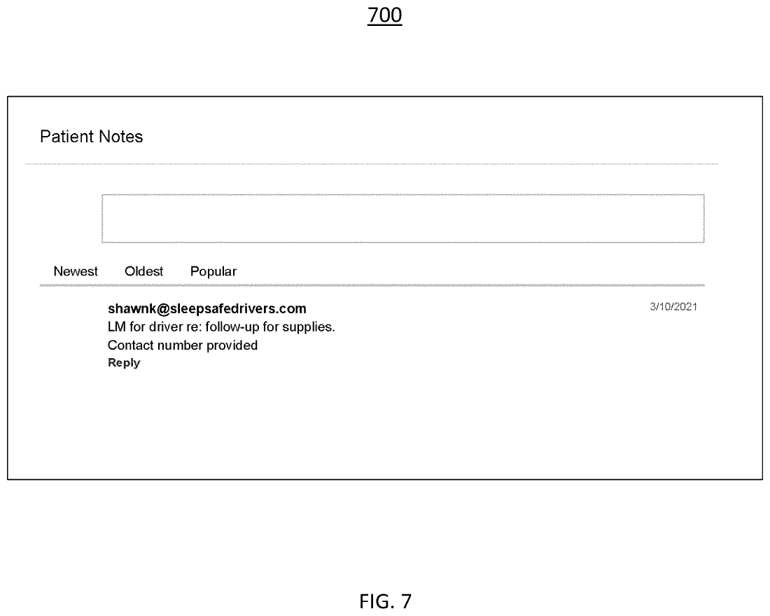

FIG. 7 illustrates a form 700 for entering text associated with a patient consistent with implementations of the current subject matter. The text can be entered by a user (e.g., a therapist, employer, etc.) on terminal 170, and sent to server 120 for storage in the compliance database 130. The text can be later retrieved from the compliance database 130 and reviewed along with the rest of the comparison data of the patient.

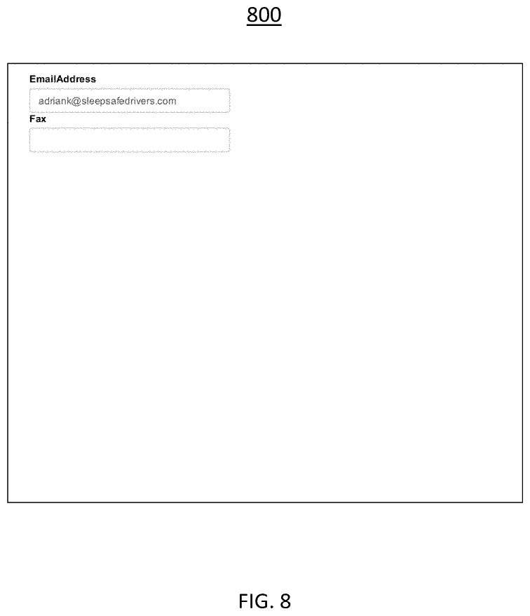

FIG. 8 illustrates an email address output form 800 consistent with implementations of the current subject matter. A user can request a compliance report on one of the patients. The compliance report is then sent to the email address and/or the fax number that is provided in the output email address form 800.

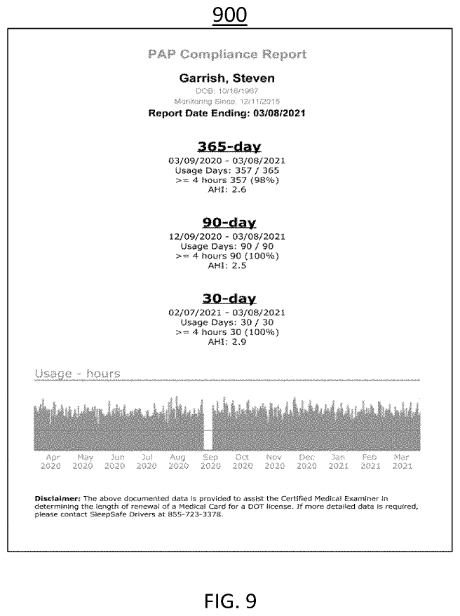

FIG. 9 illustrates a compliance report output form 900 consistent with implementations of the current subject matter. The compliance report output form 900 includes the name, DOB, setup date, report end date, specific interval compliance data, and/or the like. For each interval, the number of days using the CPAP system, the number of days using the CPAP device for greater than or equal to a specified time period (e.g., 4 hours), and the apnea hypopnea index (AHI). At the bottom of the compliance report output form 900 is a bar graph representing the hours of usage over a one-year interval.

While the integrated data compliance monitoring platform may be of particular interest and value in conjunction with CPAP system data, it should be recognized that the integrated data compliance monitoring platform is suitable for other uses. The foregoing detailed description has been given for clearness of understanding only and no unnecessary limitations should be understood there from as modifications will be obvious to those skilled in the art.

While described in connection with specific embodiments thereof, it will be understood that the principles described herein is capable of further modifications and this application is intended to cover any variations, uses, or adaptations following, in general, the principles disclosed herein and including such departures from the present disclosure as come within known or customary practice within the art to which the technology pertains and as may be applied to the essential features hereinbefore set forth and as follows in the scope of the appended claims.

1. A method for integrating data and compliance monitoring, the method comprising:
loading an application programming interface (API) preselected to generate a query;
sending the query via a network to a database requesting data for a patient;
receiving the data for the patient from the database via the network;
storing the data associated with the patient in a compliance database;
comparing the data for the patient to a rule set to determine a compliance measurement; and
generating an output, the output including the compliance measurement.

2. The method of claim 1, wherein the query contains a patient ID number, a patient name, a date, a date range, a password, a device ID, and/or an authentication token.

3. The method of claim 1, wherein the wherein the query contains a request for device usage, activation date, activation status, and/or employer name.

4. The method of claim 1, wherein the query occurs at a specified interval.

5. (canceled)

6. The method of claim 1, wherein the data contains a patient ID number, a patient name, a date, a date range, a password, a device ID, an authentication token, device usage, activation date, activation status, and/or employer name.

7. The method of claim 1, wherein the data is encrypted prior to receiving the data for the patient from the database via the network.

8. The method of claim 1, wherein the database is a remote database accessible via the network.

9. The method of claim 1, wherein the data is encrypted when the data associated with the patient is stored in the compliance database.

10. (canceled)

11. The method of claim 9, wherein the compliance database is HIPAA compliant.

12. The method of claim 1, wherein the compliance measurement a pass or fail value based on a minimum threshold.

13. The method of claim 1, wherein the output is a text message or an email message.

14. (canceled)

15. The method of claim 1, wherein the output is rendered on a graphical user interface.

16. (canceled)

17. The method of claim 1, wherein the output is an automated resupply call or a report.

18. (canceled)

19. The method of claim 17, wherein the report includes data associated with a plurality of patients.

20. The method of claim 1, wherein the compliance measurement is date and/or time stamped.

21. The method of claim 1, wherein when the data includes a null value, the output indicates missing data.

22. The method of claim 1, further comprising:
entering text associated with the patient; and
storing the text in the compliance database.

23. The method of claim 1, further comprising:
receiving data from a piece of machinery, the piece of machinery being associated with the patient.

24. The method of claim 23, further comprising:
storing data from the piece of machinery in the compliance database.

25. (canceled)

26. The method of claim 24, wherein the piece of machinery is a vehicle and the vehicle has a gross vehicle weight rating of 26,001 or more pounds.

27.-105. (canceled)
